(19) United States
(12) Patent Application Publication
(10) Pub. No.: US 2003/0127099 A1
(43) Pub. Date: Jul. 10, 2003

(54) Demand valve which can be used in oxygen therapy

(76) Inventors: Matthieu Meneuvrier, Saint-Malo De Guersac (FR); Christian Bleys, (US); Stephane Pingret, (US)

Correspondence Address: Linda K. Russell, Air Liquide, Suite 1800, 2700 Post Oak Blvd., Houston, TX, 77506, US

(21) Appl. No.: 10/338,553

(22) Filed: Jan. 8, 2003

(30) Foreign Application Priority Data
Jan. 8, 2002 (FR) 0200150

(51) Int. Cl. A61M 16/00

(52) U.S. Cl. 128/204.26; 128/205.24

(57) ABSTRACT

Gas delivery valve, in particular of the demand-valve type, having a valve body (1) in which is made a main passage (11) for routing gas comprising a gas inlet orifice (12) and a gas outlet orifice (13), valve means (33) arranged on the internal passage (11) for routing gas, between the said gas inlet orifice (12) and outlet orifice (13), making it possible to control the flow of gas in the said main internal passage (11) for routing gas, a line (14) for detecting a pneumatic vacuum co-operating with the said valve means (33) in order to allow, at least temporarily, the flow of gas in the said main internal passage (11) for routing gas between the said inlet orifice (12) and outlet orifice (13). The valve further comprises a secondary passage (8) for routing gas which is fluidically connected to the main gas routing passage (11) upstream (11a) and downstream (11b) of the said valve means (33). Assembly formed by a flow meter (2) having an outlet connection (3) to which such a valve is securely connected and the use of this valve or of this assembly in oxygen therapy.

The present invention relates to a demand valve which can be used in patient oxygen therapy and, more specifically, such a valve equipped with a system for rapid connection to the gas outlet of a flow meter with a flow rate selector or an equivalent system making it possible to deliver a specific gas flow rate, or a continuous flow, the said rapid connection having at least two connection positions.

Some people suffer from respiratory insufficiency making it necessary to have an additional oxygen supply so as to increase the proportion of oxygen, present in the air or the gas that they breathe, beyond the oxygen content which is usually present therein.

To do this, it is usual to deliver, to the upper airways of these people, oxygen by means of gas feed systems, which may be of several different models, such as breathing masks or devices of the respiratory-clip type.

To this end, mention may be made, for example, of documents EP-A-099283, U.S. Pat. No. 4,648,395, DE-A-4309923, EP-A-991460 and EP-A-941136.

However, the delivery of oxygen to the respiratory airways of a patient may be carried out either continuously, although this generates gas losses, or in a manner which is synchronized with the patient's respiratory phases, that is to say solely during the inspiration phases.

Various solutions have already been proposed to synchronize the delivery of gas with the patient's inspiration phases.

Some of these operate by means of sensors or other electrical or electronic devices which detect the respiratory activity of the patient in the respiratory mask or by other means. By way of example, mention may be made of document EP-A-998318 which describes a control based on monitoring the oxygen content of the blood or of document WO-01-43806 which describes a device for delivering gaseous NO to a patient, which is controlled by a microprocessor unit.

Conversely, other much simpler solutions operate in a completely pneumatic mode. In this case, it is the variations in gas pressure inspired or expired by the patient which are used to determine the times at which the gas should be delivered to the patient.

Among the existing pneumatic devices, those most commonly used are demand valves, also called “economizer” valves, which control the supply of gas to the patient such that the gas is only delivered when the patient requires it, that is to say when he starts to inspire, which makes it possible to prevent gas being wasted, when the patient has no need of it.

Moreover, mention may be made of the following documents:

EP-A-937640 relating to a gas regulator which can be used in diving, itself also having a bypass on its main line serving to facilitate the diver's breathing.

WO-A-90/01965 relating to a valve system which can be used in oxygen therapy apparatus with sequential delivery of gas, during respiratory phases. This system has two gas lines, one serving to deliver the gas in the form of “pulses”, the other serving to convey the gas continuously. The delivery of the gas to one or other of these lines is controlled by means of a manual valve which can be moved between two stable positions in which the inlets of the two gas lines are alternately shut off or opened.

FR-A-2763382 relating to a gas regulator with a safety device having a bypass line intended to be used under severe conditions: very low pressures and highly polluted environments (nuclear, bacteriological or chemical pollution).

FR-A-2813799 relating to an economizer valve whose novelty appears to relate to the use of a membrane in order to control the gas flow. This document specifies that the device has no bypass passage connecting the feed passage to the delivery chamber.

U.S. Pat. No. 2,328,214 relating to an oxygen regulator.

However, the valves proposed by these documents are not ideal since none of these valves solves the problem of continuous delivery of gas, in particular in the event of breakdown of the internal mechanism, without having to disconnect the outlet conduit of the valve in order to reconnect it directly to the outlet connection of the gas source.

In other words, the problem which is presented is that in the event of breakdown or defect in their internal mechanism, the known economizer valves do not have an easy means for delivering the gas in continuous mode. Specifically, at present, in the event of breakdown or defect of the system, it is necessary to disconnect the outlet nozzle of the economizer valve in order to reconnect it directly to the flow meter outlet or the source of flow.

The aim of the invention is therefore to solve this problem by providing an improved valve making it possible to obtain a continuous delivery of the gas, in particular in the event of breakdown or defect in its internal mechanism, without having to disconnect the outlet conduit of the valve in order to reconnect it directly to the outlet connection of the gas source.

The solution proposed by the invention therefore relates to a gas delivery valve, in particular of the demand-valve or economizer-valve type, having a valve body in which is made

a main passage for routing gas comprising a gas inlet orifice and a gas outlet orifice,

valve means arranged on the internal passage for routing gas, between the said gas inlet orifice and outlet orifice, making it possible to control the flow of gas in the said main internal passage for routing gas,

a line for detecting a pneumatic vacuum co-operating with the said valve means in order to allow, at least temporarily, the flow of gas in the said main internal passage for routing gas between the said inlet orifice and outlet orifice,

a secondary passage for routing gas which is fluidically connected to the main gas routing passage upstream and downstream of the said valve means, characterized in that it comprises connection means making it possible to connect the said valve body fluidically and securely to a gas feed member so that the said main gas routing passage can be fed with gas via the said gas inlet orifice, the connection means comprising a sleeve piece which can be moved in translation in the said housing forming the inlet of the main gas routing passage, the said sleeve piece having a central recess making it possible for the gas to pass through the said sleeve piece, and the moveable sleeve piece being capable of taking at least:

a first stable position for continuous gas delivery, in which the gas is routed via the secondary gas routing passage in order to be delivered continuously via the gas outlet orifice, and

a second stable position for discontinuous gas delivery, in which the gas passes through the valve means so as to be delivered discontinuously via the gas outlet orifice and only when a vacuum appears in the vacuum detection line.

Depending on the case, the valve of the invention may comprise one or more of the following technical features:

a housing is made on the main gas routing passage, one wall of the said housing bearing the gas inlet orifice and at least part of the connection means being located in the said housing or in the wall of the said housing.

the sleeve piece bears sealing means on its outer peripheral wall, preferably the sealing means are two O-ring seals spaced apart from each other.

when the sleeve piece is in its second stable position, the sealing means borne by the outer peripheral wall of the sleeve piece prevent any introduction of gas into the upstream inlet of the secondary gas routing passage which is fluidically connected to the main gas routing passage upstream of the said valve means.

a holding mechanism allows the sleeve piece to be held in its first stable position and/or in its second stable position, preferably the release mechanism comprises a finger-actuated button and a return spring allowing it to return to its position when it is not actuated.

a release mechanism co-operating with the holding mechanism allows the sleeve piece to be released when it is in the first stable position and/or in the second stable position, preferably the release mechanism has a finger-actuated button.

an elastic means located in the main gas routing passage exerts a thrust force on the sleeve piece in the direction tending to move away from the upstream inlet of the secondary passage.

According to another aspect, the invention also relates to an assembly formed by a flow meter comprising an outlet connection to which a valve according to the invention is connected.

The invention will now be better understood by means of the detailed explanation given hereinbelow with reference to the appended figures.

FIG. 1 shows, schematically, in sectional view, an economizer valve 1 according to the invention which is designed to be connected to the outlet connection or olive 3 of a flow meter 2 mounted on the outlet of a pressurized gas source, such as a bottle of oxygen or a line. The valve is connected mainly via a moveable sleeve piece 4 or moveable barrel having a mechanism allowing the flow meter to be fastened to the olive 3. A spring 5 or the like, together with a stop (not shown) allow the barrel 4 to be kept in the desired position.

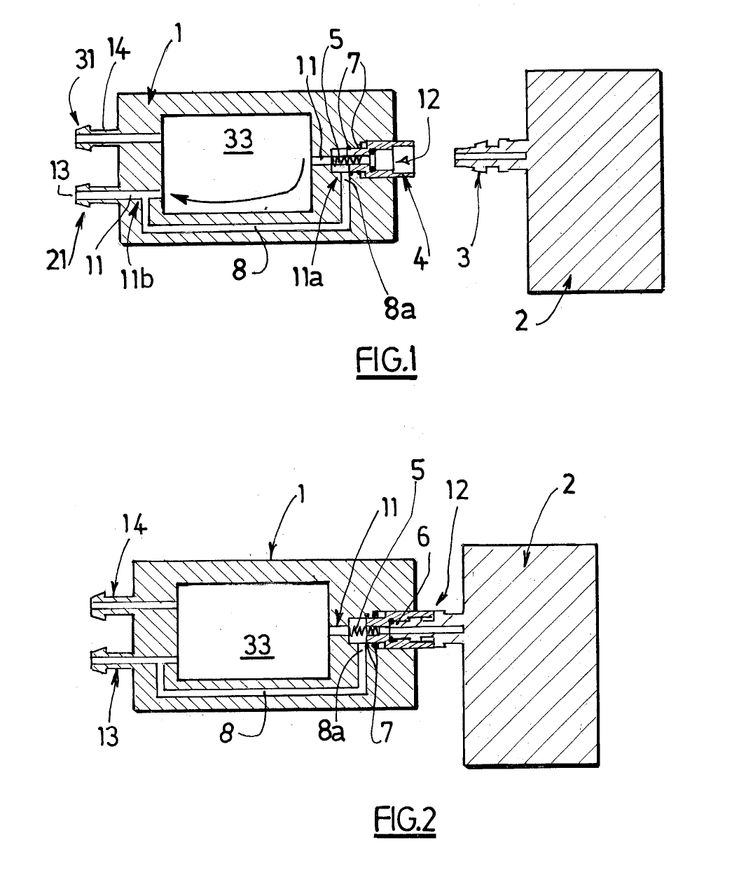

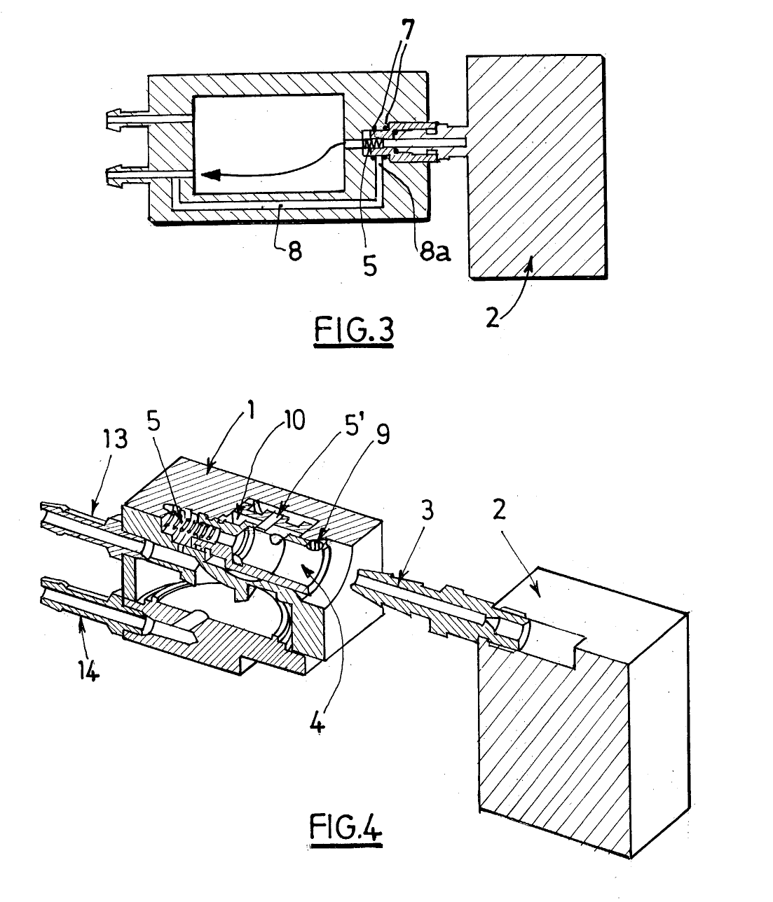

According to the invention, the valve 1 has at least two stable connection positions, that is an intermediate position which allows the assembly connected to the connection to operate in continuous mode (FIG. 2) and a position called “stop” position which implements the economizer mode of the valve 1 (FIG. 3), providing a discontinuous delivery of the gas depending on the patient's demand for gas.

As shown in FIG. 2, in the intermediate position, the economizer valve slides over the olive of the flow meter which is housed in the barrel 4, the seal between these two parts being achieved by an O-ring 6. Similarly, the seal between the barrel 4 and the body 1 of the economizer is achieved by two other O-ring seals 7.

In this position, the gas coming from the flow meter passes through an internal bypass channel 8 which communicates directly with the outlet called “power” outlet of the valve, which feeds the patient's airways with gas. The presence of this passage 8 makes it possible to prevent the gas passing into the mechanism of the economizer valve, which ensures the unit operates in continuous flow rate mode.

Moreover, as shown in FIG. 3, if an additional pressure force is exerted by an operator (user or medical personnel) on the body 1 of the economizer valve, in the direction going from the axis of the olive to the flow meter, then the moveable barrel 4 is moved in translation into another stable position in which the inlet of the channel 8 is obstructed by the two seals 7.

In this position called “stop” position, the gas coming from the flow meter has to pass through the economizer valve mechanism and the valve then plays its conventional role, that is to say that a flow of gas will be delivered to the patient only when a vacuum is created at the outlet called “detection” outlet of the valve body 1, the said protection outlet being pneumatically connected to the patient's airways, for example by means of a flexible gas hose. In other words, operation of the demand valve is in “economy” mode or on demand. In all cases, the gas always comes out of the outlet called “power” outlet of the valve body 1.

FIGS. 4 to 6 show an exemplary embodiment of the valve of the invention in partial view in three dimensions and in section allowing the operation of the mechanism for fastening the valve to the olive or connection 3 of the flow meter 2 to be understood.

As can be seen, the valve barrel 4 is held on a stop 5′ secured to a button 10 by the spring 5. In this position, the three balls 9 are free in their respective housing in the barrel 4, these balls 9 serving to provide the fastening for the olive 3 to the barrel 4, when the latter is connected to the connection 2.

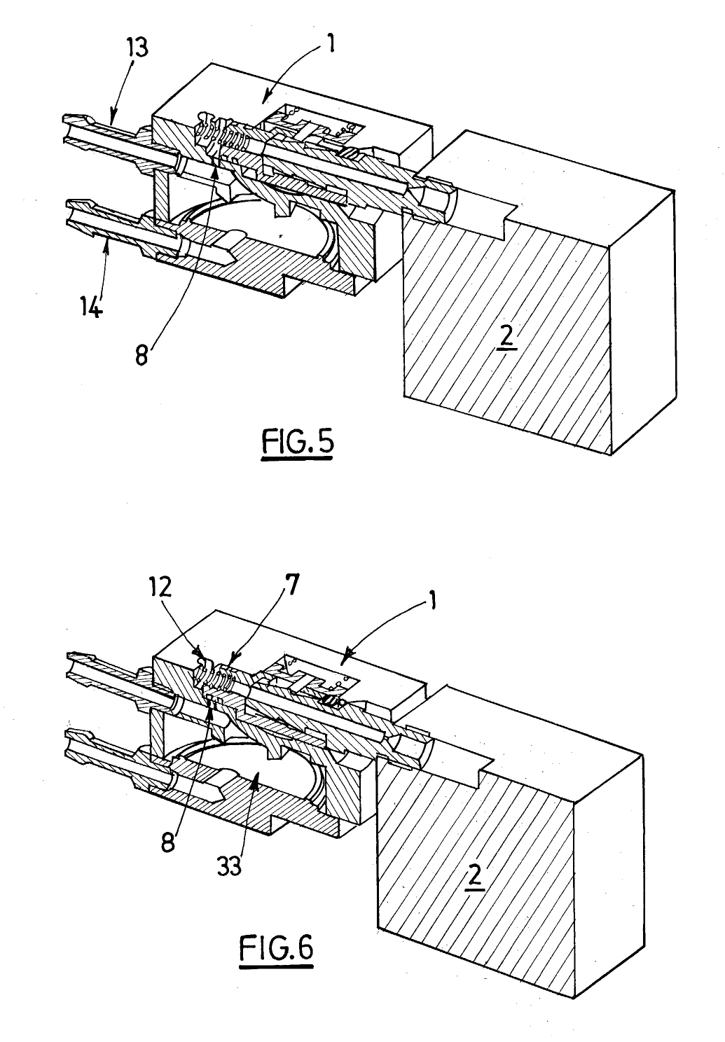

In the intermediate position (FIG. 5), the gas passes through the bypass channel 8 then through the “power” outlet. In this position, the three balls 9 are restricted to the groove of the olive 3 of the flow meter 2, which ensures the valve is fastened thereon.

Moreover, when the demand valve is in the stop position (FIG. 6), the channel 8 is obstructed by the two O-ring seals 7, the gas then passes through the conduit 12 which communicates with the demand valve mechanism 33. The demand valve then operates in economizer mode.

The stable position in each of the intermediate or stop positions is obtained by virtue of positioning the three balls 9 which co-operate with a slope made in the housing accommodating the barrel 4. When the barrel takes a rear position by axial thrust, it has the effect of bringing the balls closer to the axis of the barrel, these latter then being housed in the groove made in the olive 3, thereby retaining it inside the barrel. Locking is obtained by returning the button 10 by means of an adjacent axial spring placed at its end. Conversely, the assembly is unlocked by simple finger pressure on the button 10 allowing the barrel to drop and the release of the bores to a larger external diameter, which has the effect of releasing the groove made in the olive 3, the latter thus being able to be detached from the moveable barrel 4.

According to the invention, the moveable sleeve piece 4 in the form of a barrel is therefore able to take at least two stable positions, that is:

a first stable position in which the gas may successively pass through, in a continuous manner, the central recess of the sleeve piece, the secondary gas routing passage 8 acting as a bypass channel and the downstream portion of the main gas routing passage located downstream of the said valve means 33 in order to finish at the gas outlet orifice, that is to say the power outlet, and

a second stable position in which the gas may successively pass through the central recess of the sleeve piece 4, the valve means and the downstream portion of the main gas routing passage located downstream of the said valve means to finish at the gas outlet orifice, and in which the upstream inlet of the secondary gas routing passage 8 fluidically connected to the main gas routing passage upstream of the said valve means is closed off, preferably the upstream inlet of the secondary passage is closed off by the sealing means, such as O-ring seals, borne by the outer peripheral wall of the sleeve piece 4.

The rapid connector system having two positions for connection to the gas source or flow meter makes it possible, due to its position, for the economizer valve to operate in continuous mode or in discontinuous mode.

The valve of the invention or a valve/flow meter assembly can be used in oxygen therapy.

1. Gas delivery valve, in particular of the demand-valve type, having a valve body (1) in which is made:
a main passage (11) for routing gas comprising a gas inlet orifice (12) and a gas outlet orifice (13),
valve means (33) arranged on the internal passage (11) for routing gas, between the said gas inlet orifice (12) and outlet orifice (13), making it possible to control the flow of gas in the said main internal passage (11) for routing gas,
a line (14) for detecting a pneumatic vacuum co-operating with the said valve means (33) in order to allow, at least temporarily, the flow of gas in the said main internal passage (11) for routing gas between the said inlet orifice (12) and outlet orifice (13),
a secondary passage (8) for routing gas which is fluidically connected to the main gas routing passage (11) upstream (11a) and downstream (11b) of the said valve means (33),
characterized in that it comprises connection means (4, 9, 5′, 10) making it possible to connect the said valve body (1) fluidically and securely to a gas feed member (3) so that the said main gas routing passage (11) can be fed with gas via the said gas inlet orifice (12), the connection means (4, 9, 5′ 10) comprising a sleeve piece (4) which can be moved in translation in the said housing forming the inlet (12) of the main gas routing passage (11), the said sleeve piece (4) having a central recess making it possible for the gas to pass through the said sleeve piece (4), and the moveable sleeve piece (4) being capable of taking at least:
a first stable position for continuous gas delivery, in which the gas is routed via the secondary gas routing passage (8) in order to be delivered continuously via the gas outlet orifice (13), and
a second stable position for discontinuous gas delivery, in which the gas passes through the valve means (33) so as to be delivered discontinuously via the gas outlet orifice (12) and only when a vacuum appears in the vacuum detection line (14).

2. Valve according to claim 1, characterized in that a housing is made on the main gas routing passage (11), one wall of the said housing bearing the gas inlet orifice (12) and at least part of the connection means (4, 9, 5′, 10) being located in the said housing or in the wall of the said housing.

3. Valve according to either of claims 1 and 2, characterized in that the sleeve piece (4) bears sealing means (7) on its outer peripheral wall.

4. Valve according to one of claims 1 to 3, characterized in that the sealing means (7) are two O-ring seals spaced apart from each other.

5. Valve according to one of claims 1 to 4, characterized in that, when the sleeve piece (4) is in its second stable position, the sealing means (7) borne by the outer peripheral wall of the sleeve piece (4) prevent any introduction of gas into the upstream inlet (8a) of the secondary gas routing passage (8) which is fluidically connected to the main gas routing passage (11) upstream of the said valve means (33).

6. Valve according to one of claims 1 to 5, characterized in that a holding mechanism allows the sleeve piece (4) to be held in its first stable position and/or in its second stable position.

7. Valve according to one of claims 1 to 6, characterized in that the release mechanism comprises a finger-actuated button (10) and a return spring allowing it to return to its position when it is not actuated.

8. Valve according to one of claims 1 to 7, characterized in that a release mechanism co-operating with the holding mechanism allows the sleeve piece (4) to be released when it is in the first stable position and/or in the second stable position.

9. Valve according to one of claims 1 to 8, characterized in that the release mechanism has a finger-actuated button (10).

10. Valve according to one of claims 1 to 9, characterized in that an elastic means (5) located in the main gas routing passage (11) exerts a thrust force on the sleeve piece (4) in the direction tending to move away from the upstream inlet (8a) of the secondary passage (8).

11. Unit formed by a flow meter (2) comprising an outlet connection (3) to which a valve according to one of claims 1 to 10 is securely connected.
